(19) United States
(12) Patent Application Publication
(10) Pub. No.: US 2017/0071514 A1
(43) Pub. Date: Mar. 16, 2017

(54) MEASUREMENT APPARATUS, MEASUREMENT SYSTEM, MEASUREMENT METHOD, AND ELECTRONIC DEVICE PROVIDED WITH MEASUREMENT APPARATUS

(71) Applicant: KYOCERA Corporation, Kyoto (JP)

(72) Inventor: Hideki HIDAKA, Hino-shi (JP)

(73) Assignee: KYOCERA Corporation, Kyoto (JP)

(21) Appl. No.: 15/123,500

(22) PCT Filed: Mar. 12, 2015
(86) PCT No.: PCT/JP2015/001383

§ 371 Date: Sep. 2, 2016

(30) Foreign Application Priority Data
Mar. 26, 2014 (JP) 2014-064134

(51) Int. Cl. A61B 5/145; A61B 5/00

(52) U.S. Cl. CPC A61B 5/14532; A61B 5/7405; A61B 5/742; A61B 5/7455

(57) ABSTRACT

A measurement apparatus includes a measurement unit, a light emitter configured to emit light onto a test site of a subject, a light receiver configured to receive reflected light from the test site, a pressure detector configured to detect pressure on the measurement unit, and a calculator configured to calculate a concentration of a predetermined component in blood of the subject contacting the measurement unit based on output of the light receiver when the pressure is within a first range and on output of the light receiver when the pressure is within a second range.

### CROSS-REFERENCE TO RELATED APPLICATION

This application claims priority to and the benefit of Japanese Patent Application No. 2014-064134 filed Mar. 26, 2014, the entire contents of which are incorporated herein by reference.

### TECHNICAL FIELD

This disclosure relates to a measurement apparatus, a measurement system, a measurement method, and an electronic device provided with a measurement apparatus.

### BACKGROUND

A variety of measurement methods for measuring the concentration of a component included in the blood of a subject are known. In recent years, non-invasive measurement methods that perform measurement without harming the subject have been attracting attention in order to reduce the burden on the subject during measurement of the concentration of a component in blood. For example, there is a non-invasive method for measuring a component included in the blood of a living body.

### SUMMARY

One measurement apparatus according to this disclosure is a measurement apparatus including:

a measurement unit;

a light emitter configured to emit light onto a test site of a subject;

a light receiver configured to receive reflected light from the test site;

a pressure detector configured to detect pressure on the measurement unit; and

a calculator configured to calculate a concentration of a predetermined component in blood of the subject contacting the measurement unit based on output of the light receiver when the pressure is within a first range and on output of the light receiver when the pressure is within a second range.

One measurement system according to this disclosure is a measurement system including:

a measurement terminal comprising a light emitter configured to emit light onto a test site of a subject, a light receiver configured to receive reflected light from the test site, and a pressure detector configured to detect pressure on a measurement unit; and

a calculator connected to the measurement terminal over a network and configured to calculate a concentration of a predetermined component in blood of the subject contacting the measurement unit based on output of the light receiver when the pressure is within a first range and on output of the light receiver when the pressure is within a second range.

One measurement method according to this disclosure is a method for measuring including:

emitting light onto a test site of a subject;

receiving reflected light from the test site;

detecting pressure on a measurement unit; and

calculating the concentration of a predetermined component in blood of the subject contacting the measurement unit based on output of the reflected light received when the pressure is within a first range and on output of the reflected light received when the pressure is within a second range.

### BRIEF DESCRIPTION OF THE DRAWINGS

In the accompanying drawings:

FIG. 1 is a block diagram schematically illustrating the structure of a measurement apparatus according to one of the embodiments of this disclosure;

FIGS. 2A and 2B illustrate states in which the subject is pressed against the measurement unit;

FIGS. 3A and 3B illustrate examples of reflected light that is received by a light receiver;

FIGS. 4A and 4B illustrate an example of an electronic device in which the measurement apparatus of FIG. 1 is mounted; and

FIG. 5 illustrates an example of processing for measurement of glucose concentration performed by the measurement apparatus of FIG. 1.

### DETAILED DESCRIPTION

The following describes one of the disclosed embodiments in detail with reference to the drawings.

A tourniquet needs to be used to stop the flow of blood in the body's finger. Therefore, when the user wishes to measure the concentration of a predetermined component included in the user's own blood, the user needs to prepare a tourniquet to stop the blood flow, which is not convenient for the user.

Therefore, it would be helpful to provide a measurement apparatus, a measurement system, a measurement method, and an electronic device provided with a measurement apparatus that are highly convenient.

According to this disclosure, a highly convenient measurement apparatus, measurement system, measurement method, and electronic device provided with a measurement apparatus can be achieved.

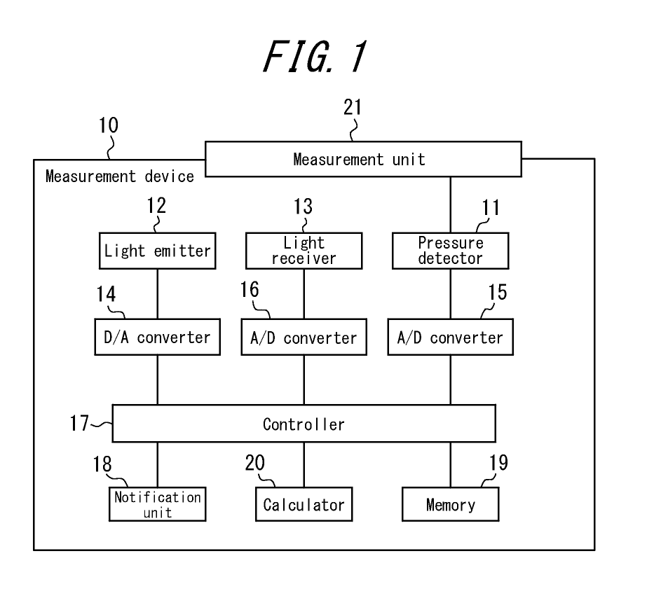

FIG. 1 is a block diagram schematically illustrating the structure of a measurement apparatus according to one of the disclosed embodiments. The measurement apparatus 10 includes a pressure detector 11, a light emitter 12, a light receiver 13, a D/A converter 14, A/D converters 15 and 16, a controller 17, a notification unit 18, a memory 19, a calculator 20, and a measurement unit 21.

As biometric information, the measurement apparatus 10 measures the concentration of a predetermined component in the blood of a living body (subject). Upon the user contacting a test site, such as a finger, to the measurement unit 21 of the measurement apparatus 10, the measurement apparatus 10 emits light (measurement light) onto the test site. Based on the reflected light (detected light) from a component that is targeted for measurement and included in a capillary at the test site, the measurement apparatus 10 measures the concentration of a predetermined component. The measurement apparatus 10 can measure concentration based on the Raman spectrum from the component targeted for measurement. The measurement apparatus 10 performs measurement when the pressure received from the test site is within a predetermined range. The predetermined range may be the range included between a predetermined upper limit and lower limit or may be a range that is at least a predetermined value or at most a predetermined value. The predetermined component may be any component included in blood. In this disclosure, the predetermined component is described below as being glucose, for example, and the measurement apparatus 10 is described as measuring the glucose concentration in the blood of the subject.

The optical absorption spectrum of glucose resembles the optical absorption spectrum of moisture. Therefore, if the measurement apparatus 10 emits light onto the test site when the test site is in a state of contact with the measurement unit 21 and receives reflected light, the reflected light includes both reflected light from glucose and reflected light from moisture included at the test site.

To address this issue, the measurement apparatus 10 measures first and second reflected light respectively in two states: a state in which the test site is touching the measurement unit 21, i.e. a state in which pressure on the measurement unit 21 is low, and a state in which the test site is pressed strongly against the measurement unit 21, i.e. a state in which pressure on the measurement unit 21 is high. Based on the measurement results of the first and second reflected light, the glucose concentration is measured.

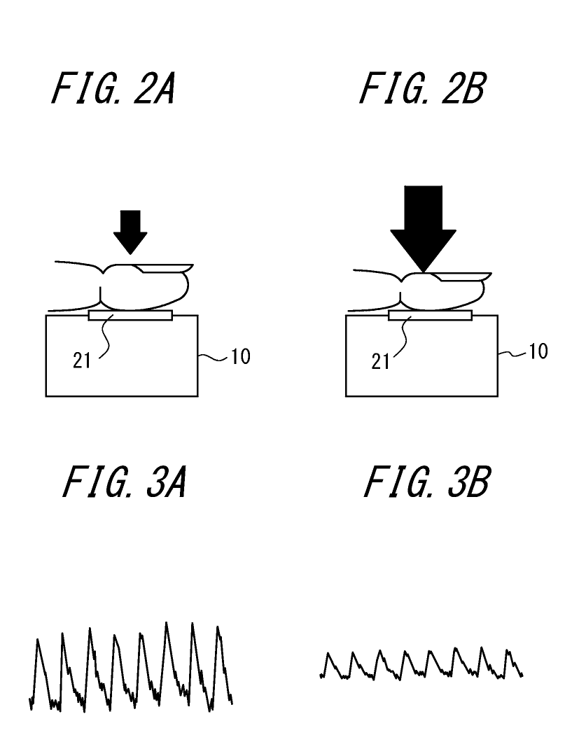

FIGS. 2A and 2B illustrate states in which the finger, which is the test site of the subject, is pressed against the measurement apparatus 10. FIG. 2A illustrates a state in which pressure on the measurement unit 21 from the finger is low. At this time, since a capillary is in a state of not being pressed (first state), glucose flows in the capillary. Therefore, the first reflected light received by the measurement apparatus 10 includes reflected light from glucose and reflected light from moisture.

FIG. 3A illustrates an example of the first reflected light. The first reflected light illustrated in FIG. 3A includes reflected light from glucose and reflected light from moisture. As illustrated in FIG. 3A, the reflected light represents reflection from glucose and moisture included in the capillary, and the intensity of the reflected light varies in conjunction with contraction of blood vessels due to the rhythm of blood flow.

FIG. 2B illustrates a state in which pressure on the measurement unit 21 from the finger is high. At this time, the capillary at the test site is in a crushed state (second state), and glucose does not flow in the capillary. Accordingly, the second reflected light received by the measurement apparatus 10 at this time is reflected light from moisture and does not include reflected light from glucose.

FIG. 3B illustrates an example of the second reflected light. The second reflected light illustrated in FIG. 3B includes reflected light from moisture and does not include reflected light from glucose. As in FIG. 3A, in FIG. 3B as well, the reflected light represents reflection from moisture included in the capillary, and the intensity of the reflected light varies in conjunction with contraction of blood vessels due to the rhythm of blood flow. In FIG. 3B, however, as compared to FIG. 3A, the reflection intensity at the peak of the reflected light is reduced by an amount corresponding to the non-inclusion of reflected light from glucose. In other words, the difference in reflection intensity between FIGS. 3A and 3B corresponds to reflected light from glucose.

The measurement apparatus 10 detects pressure on the measurement unit 21 and measures the first and second reflected light respectively when the capillary is in the first state and in the second state. Based on the difference between the measurement results of the first and second reflected light measured in this way, the measurement apparatus 10 measures the glucose concentration in the blood.

Referring again to FIG. 1, the pressure detector 11 detects the pressure on the measurement unit 21. The pressure detector 11 may, for example, be configured using a piezoelectric element. The pressure detector 11 is connected to the controller 17 via the A/D converter 15 and transmits a detected pressure signal to the controller 17 after conversion to a digital signal by the A/D converter 15.

The light emitter 12 emits light (measurement light) onto the test site of the subject. The light emitter 12 may, for example, be a laser light source that emits laser light as the measurement light, the laser light having a predetermined wavelength that can detect the component that is targeted for measurement. In this embodiment, the light emitter 12 is described below as being a Laser Diode (LD) that emits infrared light. The light emitter 12 is connected to the controller 17 via the D/A converter 14 and emits infrared light based on a signal received from the controller 17 via the D/A converter 14.

The light emitter 12 may be configured to operate only when measuring reflected light in the first and second states. In other words, the light emitter 12 may be configured to emit light when the pressure on the measurement unit 21 is in a predetermined range. With this configuration, emission of laser light from the light emitter 12 can be prevented when measurements are not being taken.

By the light emitter 12 irradiating the test site with measurement light, the light receiver 13 receives reflected light that is reflected at the test site. Therefore, as more of the component that is targeted for measurement is present at the test site, the intensity of reflected light that is received by the light receiver 13 increases. The light receiver 13 may, for example, be configured using a photodiode (PD). The light receiver 13 is connected to the controller 17 via the A/D converter 16 and transmits a photoelectric conversion signal of the received reflected light to the controller 17 after conversion of the signal from analog to digital by the A/D converter 16.

The controller 17 is a processor that, starting with the functional blocks of the measurement apparatus 10, controls and manages the measurement apparatus 10 overall. The controller 17 is configured using a processor such as a Central Processing Unit (CPU) that executes a program prescribing control procedures. Such a program may, for example, be stored in the memory 19, in an external storage medium, or the like.

The notification unit 18 notifies the user, who is the subject, of information related to the pressure on the measurement unit 21. The measurement apparatus 10 measures the glucose concentration when the pressure on the measurement unit 21 is within a predetermined range. Therefore, when the pressure is not in a predetermined range, the notification unit 18 can notify the user so as to prompt the user to apply pressure on the measurement unit 21 within the predetermined range.

When measuring the glucose concentration, the measurement apparatus 10 receives the first and second reflected light respectively in two states, i.e. the first state and the second state, as described above. Therefore, the notification unit 18 can notify the user as to whether the pressure on the measurement unit 21 is in each of the states appropriate for the measurement of glucose concentration. In greater detail, the notification unit 18 notifies the user of information on whether the pressure on the measurement unit 21 when receiving the first received light in the first state is within a range in which pressure is low enough for glucose to flow in capillaries (first range). The notification unit 18 also notifies the user of information on whether the pressure on the measurement unit 21 when receiving the second received light in the second state is within a range in which capillaries are crushed so that glucose cannot pass through blood vessels (second range).

The notification unit 18 can provide notification with any method recognizable by the user. For example, the notification unit 18 can output an error sound that informs the user of an error from a speaker provided in a mobile phone 30. The notification unit 18 can also, for example, display an error image that indicates an error on a back display provided separately from the measurement apparatus 10 on the back face of the mobile phone 30. The notification unit 18 can also notify the user of an error by, for example, emitting light with a light emitting element provided on the back face of the mobile phone 30. Furthermore, the notification unit 18 can notify the user of an error by, for example, generating vibration with an interval vibration unit, such as a vibrator or a piezoelectric element. The method of providing notification with the notification unit 18 is not limited to the above examples. The notification unit 18 may also notify the user of an error with any combination of methods.

Furthermore, the notification unit 18 can provide notification of different errors when the pressure on the measurement unit 21 is stronger than and weaker than the predetermined range. For example, when providing notification of an error by generating vibration with a vibration unit, the notification unit 18 can provide notification of an error with different vibration patterns in the cases of the pressure being stronger than and weaker than the predetermined range. By thus being notified of different errors, the user can easily recognize whether to apply more pressure or less pressure to the measurement unit 21 with the finger, thus making it easier to adjust the pressure to be within the predetermined range.

The notification unit 18 can also provide the user with notification at the start and the end of reception of detected light by the light receiver 13. By being notified of the start of light reception, the user can recognize the need to maintain the pressure state of the finger, and by being notified of the end of light reception, the user can recognize that the finger can be released from the measurement unit 21.

The memory 19 may be configured with a semiconductor memory or the like. The memory 19 stores a variety of information, programs for causing the measurement apparatus 10 to operate, and the like and also functions as a working memory. The memory 19 also stores table data indicating the correspondence relationship between i) the difference between the measurement results of the first and second reflected light measured respectively in the first state and the second state and ii) the glucose concentration in the blood. The table data are created in advance and stored in the memory 19.

The calculator 20 measures the concentration of the component targeted for measurement based on the reflected light received by the light receiver 13. In this embodiment, based on the output of the light receiver 13 when the pressure on the measurement unit 21 is within the first range and the output of the light receiver 13 when the pressure is within the second range, the calculator 20 measures the concentration of glucose with reference to the table data stored in the memory 19.

The measurement unit 21 is a portion that contacts the test site, such as a finger, in order for the user to measure biometric information. The measurement unit 21 may, for example, be configured using a plate-shaped member. The measurement unit 21 may also be configured using a member that is transparent at least with respect to the measurement light and the detected light. Upon contact with a finger or the like, the measurement unit 21 transmits the pressure on the measurement unit 21 to the pressure detector 11.

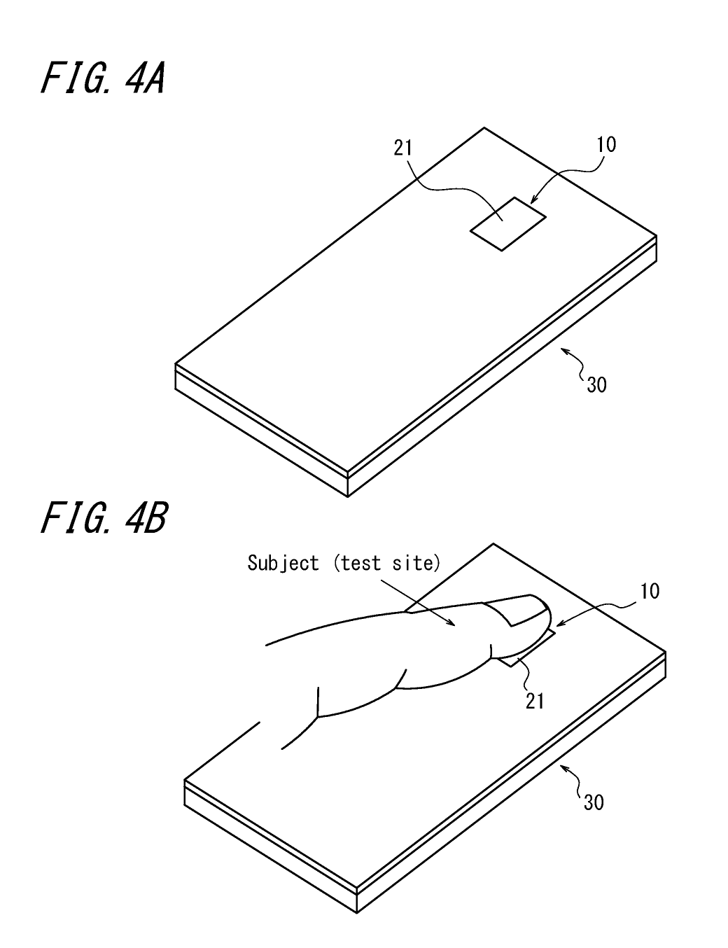

FIGS. 4A and 4B illustrate an example of an electronic device in which the measurement apparatus 10 of FIG. 1 is mounted. In this embodiment, the electronic device is the mobile phone 30, which for example is a smartphone or the like. As illustrated in FIG. 4A, the mobile phone 30 is provided with the measurement apparatus 10 on the back face. For example as illustrated in FIG. 4B, the glucose concentration is measured by pressing the pad of a finger, which is the test site, against the measurement apparatus 10 on the back face of the mobile phone 30.

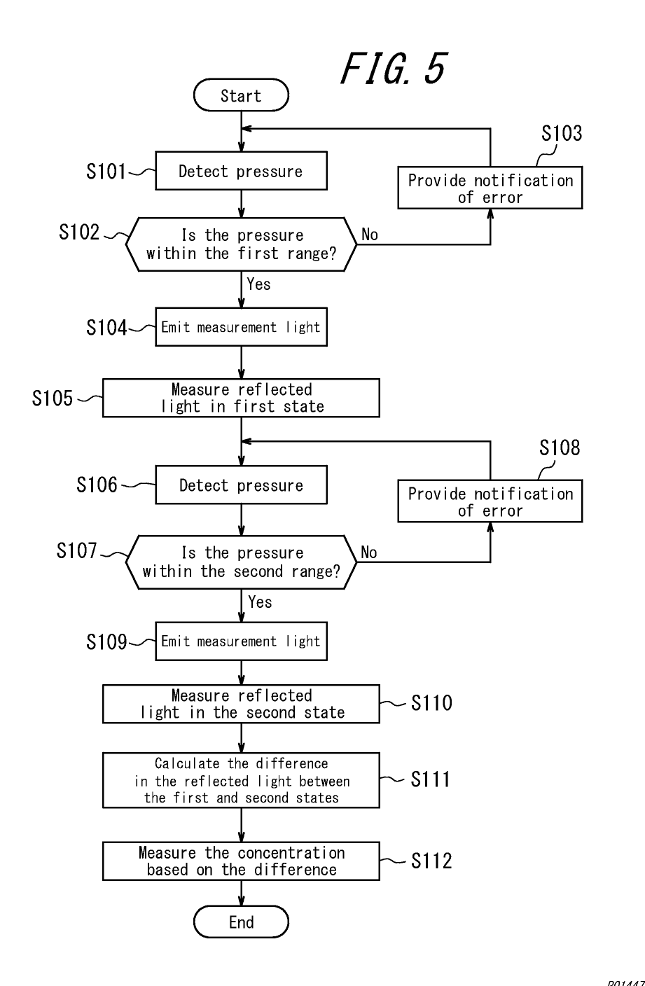

FIG. 5 illustrates an example of processing for measurement of glucose concentration performed by the measurement apparatus 10 of FIG. 1. With reference to FIG. 5, a method for measuring the glucose concentration with the measurement apparatus 10 is described.

In order to measure the glucose concentration, the user first launches a dedicated application for measuring glucose concentration. The user can, for example, launch the application for measuring glucose concentration by operating an input interface provided in the mobile phone 30. At this time, the light emitter 12 is not operating and is not emitting measurement light.

Once the application for measuring glucose concentration launches, the user presses a finger against the measurement apparatus 10 on the back face of the mobile phone 30. The user first presses the measurement unit 21 with the finger so that the pressure against the measurement unit 21 is within a first range that is low enough for glucose to flow in capillaries. The controller 17 detects the pressure on the measurement unit 21 with the pressure detector 11 (step S101). The controller 17 then judges whether the detected pressure is within the first range (step S102).

When the detected pressure is not within the first range, i.e. when the detected pressure is weaker or stronger than the pressure included within the first range (step S102: No), the notification unit 18 in the measurement apparatus 10 notifies the user of an error indicating that the pressure is not within the first range (step S103). At this time, the notification unit 18 may provide notification with an image display or by audio for the user to apply a stronger or weaker pressure so that the detected pressure enters the first range.

After recognizing the error from the notification unit 18, the user adjusts the pressure from the finger on the measurement unit 21. The user adjusts the pressure by weakening pressure from the finger when the pressure is stronger than the first range and strengthening pressure on the measurement unit 21 when the pressure is weaker than the first range. The pressure detector 11 then detects the pressure on the measurement unit 21 again (step S101) and judges whether the pressure is within the first range (step S102). The measurement apparatus 10 repeats processing from step S101 to step S103 until the pressure on the measurement unit 21 enters the first range.

When the controller 17 judges that the pressure on the measurement unit 21 is within the first range, i.e. that the capillary is in the first state (step S102: Yes), the controller 17 emits measurement light from the light emitter 12 (step S014). At this time, the notification unit 18 may notify the user that the pressure has entered the first range. The light receiver 13 then measures the reflected light for a predetermined time (step S105). At this time, the light receiver 13 for example receives reflected light with a waveform such as the one illustrated in FIG. 3A. Reflected light from moisture included in the test site and from glucose flowing in the capillary is included in the reflected light that is measured.

Once measurement of the reflected light in the first state is complete, the notification unit 18 notifies the user that measurement is complete. After recognizing the notification that measurement is complete, the user then presses the finger against the measurement unit 21 more strongly to adjust the capillary to be in the second state. In greater detail, as in step S101, the measurement unit 21 detects pressure on the measurement unit 21 (step S106). The measurement apparatus 10 then judges whether the detected pressure is within the second range (step S107).

When the detected pressure is not within the second range, i.e. when the detected pressure is weaker or stronger than the pressure included in the second range (step S107: No), the notification unit 18 notifies the user of an error indicating that the pressure is not within the second range (step S108). At this time, the notification unit 18 may provide notification with an image display or by audio for the user to apply a stronger or weaker pressure so that the detected pressure enters the second range.

After recognizing the error from the notification unit 18, the user adjusts the pressure from the finger on the measurement unit 21. The user adjusts the pressure by weakening pressure from the finger when the pressure is stronger than the second range and strengthening pressure on the measurement unit 21 when the pressure is weaker than the second range. The pressure detector 11 then detects the pressure on the measurement unit 21 again (step S106) and judges whether the pressure is within the second range (step S107). The measurement apparatus 10 repeats processing from step S106 to step S108 until the pressure on the measurement unit 21 enters the second range.

When the controller 17 judges that the pressure from the user's finger is within the second range, i.e. that the capillary is in the second state (step S107: Yes), the controller 17 causes measurement light to be emitted from the light emitter 12 (step S109). At this time, the notification unit 18 may notify the user that the pressure has entered the second range. The light receiver 13 then measures the reflected light for a predetermined time (step S110). At this time, the light receiver 13 for example receives reflected light with a waveform such as the one illustrated in FIG. 3B. Reflected light from moisture included in the test site is included in the reflected light that is measured, but since the capillary is crushed and glucose does not flow in the capillary at the test site, reflected light from glucose is not included in the reflected light that is measured.

Once measurement of the reflected light in the second state is complete, the notification unit 18 notifies the user that measurement is complete. The measurement apparatus 10 then calculates the difference between the measurement result for reflected light in the first state measured in step S105 and the measurement result for reflected light in the second state detected in step S110 (step S110). In greater detail, the measurement apparatus 10 calculates the difference between the photoelectric conversion signals of the reflected light received for a predetermined time for the reflected light in each of the first and second states. This difference corresponds to the amount of reflected light from glucose flowing in the capillary in the first state.

Based on the difference calculated in step S110, the calculator 20 in the measurement apparatus 10 then refers to the table data stored in the memory 19 to measure the glucose concentration in the user's blood (step S112). For example, a correspondence table between the glucose concentration measured in advance under predetermined conditions and the intensity of received Raman scattered light is stored in the table data, and based on the aforementioned difference, the calculator 20 measures the glucose concentration in the user's blood by referring to the table data.

The user can learn the measurement result by operating the mobile phone 30 and causing the measured glucose concentration to be displayed on a display, for example, provided in the mobile phone 30. When displaying the measurement result, the mobile phone 30 may, for example, also display the result of comparison with a past measurement result or display the change over time in the measurement result.

In this way, the measurement apparatus 10 according to this embodiment can measure the user's biometric information without using a tourniquet. Therefore, the user can measure biometric information simply by pressing the finger against the measurement apparatus 10. The user can thus be provided with a highly convenient measurement apparatus. Furthermore, when the pressure from the user's finger on the measurement unit 21 is outside of an appropriate range, the notification unit 18 provides notification, thereby allowing the user to adjust pressure easily to an appropriate range. Therefore, the user can easily achieve a pressure state appropriate for measurement and can smoothly measure biometric information.

When measuring the glucose concentration in the blood, the measurement apparatus 10 measures the reflected light in two states, namely states in which glucose does and does not flow in a capillary positioned at the test site, and measures the concentration based on the difference therebetween. Therefore, glucose can be extracted from a combination of water and glucose, which have a similar absorption spectrum, and the glucose concentration can be measured accurately. Moreover, based on the pressure detector 11 and the notification unit 18 provided in the measurement apparatus 10, the user can easily adjust pressure to achieve the above-described two states.

In the above embodiment, the measurement apparatus 10 has been described as measuring concentration using the Raman spectrum from a component targeted for measurement, but the method of measuring concentration in this disclosure is not limited to this method, and another method may be used instead. For example, the measurement apparatus 10 may measure the concentration based on the Rayleigh spectrum or based on both the Raman spectrum and the Rayleigh spectrum. For example, when using the Rayleigh spectrum, the measurement apparatus 10 may take advantage of the fact that for glucose, an absorption peak appears in light near a wavelength of approximately 1600 nm and may emit light near a wavelength of 1600 nm onto the test site, measure the absorption rate of the reflected light (scattered light) with respect to the irradiation light, and measure the glucose concentration with respect to predetermined table data.

This disclosure is not limited to the above embodiments, and a variety of modifications and changes are possible. For example, the functions and the like included in the various components and steps may be reordered in any logically consistent way. Furthermore, components or steps may be combined into one or divided.

Although the disclosure is based on some embodiments and the accompanying drawings, it is to be noted that various changes and modifications will be apparent to those skilled in the art based on this disclosure. Therefore, such changes and modifications may be to be understood as included within the scope of this disclosure. For example, in some functions and structural components and the like of some embodiments may be reordered in any logically consistent way. Furthermore, the functions and the structural components may be combined into some embodiments or divided from an embodiment.

For example, the arrangement of the measurement apparatus 10 in the mobile phone 30 is not limited to the examples illustrated in FIGS. 4A and 4B. The measurement apparatus 10 may, for example, be disposed in a different part of the back face of the mobile phone 30 or may be disposed on the front face, side face, or the like of the mobile phone 30.

The electronic device in which the measurement apparatus 10 is mounted is not limited to the mobile phone 30. The measurement apparatus 10 may be mounted in any type of electronic device, such as a portable music player, a laptop computer, a wristwatch, a tablet, a game device, or the like. The measurement apparatus 10 is also not limited to being mounted in an electronic device and may be used independently.

In the above embodiment, a method of measuring the glucose concentration in blood using the measurement apparatus 10 has been described, but the measurement apparatus 10 can measure the concentration of a different predetermined component. In such a case, the light emitter 12 emits, onto the test site, laser light of a predetermined wavelength that can detect the component that is targeted for measurement.

In the above embodiment, the light emitter 12 has been described as being configured to operate only when measuring reflected light in the first and second states. Similarly, the light receiver 13 may be configured to operate only when measuring reflected light in the first and second states. In other words, the light receiver 13 may be configured to receive the reflected light when the pressure on the measurement unit 21 is within the first range and the second range. By suspending the light receiver 13 when not measuring reflected light, power consumption in the measurement apparatus 10 (mobile phone 30) can be reduced.

When measuring the glucose concentration in the above embodiment, the user has been described as launching a dedicated application for measuring glucose concentration, but this disclosure is not limited to this embodiment. For example, when the measurement apparatus 10 detects pressure on the pressure detector 11, the mobile phone 30 may automatically launch the application and begin to measure pressure in the measurement apparatus 10.

In the above embodiment, all of the functions of the measurement unit 21, pressure detector 11, light emitter 12, light receiver 13, calculator 20, and the like have been described as being implemented on one terminal, but this disclosure is not limited to this embodiment. This disclosure for example includes a configuration as a measurement system in which the measurement unit 21, pressure detector 11, light emitter 12, and light receiver 13 are implemented on one terminal, and the calculator 20 is disposed on a server that is connected to the terminal by a network that is wired, wireless, or a combination of both. In this case, data related to pressure measured by the measurement unit 21 and data related to reflected light measured by the light receiver 13 are transmitted to the calculator 20 on the server via the network. The calculator 20 measures the glucose concentration based on the transmitted data and based on table data stored in a memory on the server and transmits the measurement result to the terminal. As compared to when implementing the functions of all of the measurement unit 21, pressure detector 11, light emitter 12, light receiver 13, and calculator 20 on one terminal, the terminal in this case may for example be reduced in size.

1. A measurement apparatus comprising:
a measurement unit;
a light emitter configured to emit light onto a test site of a subject;
a light receiver configured to receive reflected light from the test site;
a pressure detector configured to detect pressure on the measurement unit; and
a calculator configured to calculate a concentration of a predetermined component in blood of the subject contacting the measurement unit based on output of the light receiver when the pressure is within a first range and on output of the light receiver when the pressure is within a second range.

2. The measurement apparatus of claim 1, further comprising a notification unit configured to provide notification of information related to the pressure.

3. The measurement apparatus of claim 2, wherein the notification unit provides notification of information related to whether the pressure is within the first range and information related to whether the pressure is within the second range.

4. The measurement apparatus of claim 1, wherein the light emitter emits light when the pressure is within the first range and when the pressure is within the second range.

5. The measurement apparatus of claim 1, wherein the light receiver receives light when the pressure is within the first range and when the pressure is within the second range.

6. The measurement apparatus of claim 2, wherein the notification unit provides the notification by any one or more of output of sound, display of an image, emission of light, and output of vibration.

7. An electronic device comprising the measurement apparatus of claim 1.

8. A measurement system comprising:
a measurement terminal comprising a light emitter configured to emit light onto a test site of a subject, a light receiver configured to receive reflected light from the test site, and a pressure detector configured to detect pressure on a measurement unit; and
a calculator connected to the measurement terminal over a network and configured to calculate a concentration of a predetermined component in blood of the subject contacting the measurement unit based on output of the light receiver when the pressure is within a first range and on output of the light receiver when the pressure is within a second range.

9. A method for measuring comprising:
emitting light onto a test site of a subject;
receiving reflected light from the test site;
detecting pressure on a measurement unit; and
calculating the concentration of a predetermined component in blood of the subject contacting the measurement unit based on output of the reflected light received when the pressure is within a first range and on output of the reflected light received when the pressure is within a second range.
